(19) United States
(12) Patent Application Publication
(10) Pub. No.: US 2004/0048823 A1
(43) Pub. Date: Mar. 11, 2004

(54) Anticancer agent comprising mycolactone

(76) Inventor: Tae-Yoon Lee, Daegu (KR)

Correspondence Address: GREENBLUM & BERNSTEIN, P.L.C., 1950 ROLAND CLARKE PLACE, RESTON, VA, 20191, US

(21) Appl. No.: 10/416,513

(22) PCT Filed: Nov. 23, 2001
(86) PCT No.: PCT/KR01/02026

(30) Foreign Application Priority Data
Nov. 23, 2000 (KR) 2000/70089

(51) Int. Cl. A61K 48/00; A61K 31/366

(52) U.S. Cl. 514/44; 514/460

(57) ABSTRACT

This invention relates to an anticancer agent comprising mycolactone, which induces apoptotic death of cancer cells and also relates to inhibitors of Rb protein expression, including an antisense Rb oligonucleotide, which sensitize cancer cells to mycolactone. This invention relates further to an anticancer agent comprising both mycolactone and the inhibitors of Rb protein expression. Mycolactone induces cell death in cancers of breast, bladder, skin, stomach, liver, colon, and oral cavity, lymphoma, and leukemia via apoptosis pathway.

### FIELD OF THE INVENTION

This invention relates to an anticancer agent comprising mycolactone, which induces apoptotic death of cancer cells and also relates to inhibitors of retinoblastoma protein (hereinafter, Rb protein) expression, including an antisense Rb oligonucleotide, which sensitize cancer cells to mycolactone. This invention relates further to an anticancer agent comprising both mycolactone and the inhibitors of Rb protein expression.

### BACKGROUND OF THE INVENTION

Cancer is the second most common cause-of death, after circulatory diseases, in human both in male and female. Similarly, in Korea, the most common cause of death is circulatory disease, which is followed by cancer Korean Bureau of Statistics, Statistical Yearbook on Causes of Death, 1999).

Therefore, a variety of drugs and techniques have been developed to overcome cancers. One of the recently active studies on anticancer agent development includes exploration and improvement of anticancer molecules that induce cancer cell apoptosis, a physiological cell death mechanism.

It is generally known that a cancer (malignant tumor) is developed through excessive abnormal proliferation and growth of cells that are induced by various factors. These include exposure to chemical carcinogens, infection by oncogenic viruses, inherent genetic abnormalities, and so on. However, basically, all of these factors induce genetic abnormalities in cells.

Normal cells grow and are maintained harmoniously through the functional cross-regulation among oncogenes, tumor suppressor genes, and apoptosis-regulating genes.

In normal condition, oncogenes contribute to cell proliferation, growth, and differentiation through proper stimulation of protein synthesis and intracellular signal transduction. Oncogene activation by mutations or other mechanisms, however, contributes to the development of cancer by inducing excessive cell proliferation.

Meanwhile, tumor suppressor genes inhibit cell overgrowth and complement oncogene mutations through regulation of the cell cycle, which provide general harmony via opposite functioning to oncogenes. Cancer is developed, however, when the tumor suppressor genes are inactivated structurally, such as mutation, or functionally, through binding to some protein(s) that inhibits the function of tumor suppressor gene products.

Even in cases of genetic and functional abnormalities of oncogenes or tumor suppressor genes or their gene products, cell overgrowth is inhibited through exclusion of abnormal cells by apoptosis mechanism. Apoptosis-regulating genes are in charge of this role.

Except oncogenes, tumor suppressor genes, and apoptosis-regulating genes, there are other protection systems pursuing gene repair and signal transduction thus maintaining healthy cellular functions. Nonetheless, when some genetic defect occurs in those protection genes, cancer is developed even in the presence of these multiple protection systems.

Followings are descriptions about the current status of cancer therapeutics and anticancer agents developed and being used.

Generally, cancer therapeutics includes surgery, anticancer chemotherapy, immunotherapy, and gene therapy.

Surgery is the oldest and still an important cancer therapeutic. Cancer can be completely cured by surgery only when it is not disseminated and locally present. Thus, usually, surgery is combined with radiotherapy or anticancer chemotherapy to obtain better effect since there are micro-metastases at the time of diagnosis in more than 70% of cancer patients. That is, surgery is a method that removes a localized cancer tissue, and thus, has a limitation that it can be used only when the cancer metastasis is not present or only when curable metastasis is expected by supplementary treatment such as radiotherapy or anticancer chemotherapy.

Meanwhile, radiotherapy kills cancer cells using high-energy radioactive rays. Radioactive rays can affect both cancer and normal cells. However, there axe various methods and techniques that reduce effects to normal cells and, at the same time, increase destructive effects to cancer cells. That is, even though irradiation does not kill cancer cells immediately, it disrupts the proliferating properties of cancer cells and the non-proliferating cancer cells die at the end of their life spans. After each step of radiotherapy, the cancer size decreases since more cancer cells are killed, degraded, and excreted by blood transportation. Complications may occur due to a small portion of normal cells that are not recovered, even though, most normal cells are recovered from radiotherapy. These complications include loss of appetite, diarrhea, stomatitis, malaise, and skin problems.

The anticancer chemotherapeutic drugs (hereinafter, anticancer chemotherapeutics) have been developed from the first drug, methotrexate, which completely cured choriocarcinoma. Currently, about 50 anticancer chemotherapeutics are being used. Good effects have been reported especially in choriocarcinoma, leukemia, Wilm's tumor, Ewing's sarcoma, rhabdomyoma, retinoblastoma, lymphoma, and testis cancer by anticancer chemotherapy.

Mostly, the effect of anticancer chemotherapeutics is through binding and destructing the functions of nucleic acids. However, the problem is that anticancer chemotherapeutics do not selectively act on cancer cells. They also act on and destruct normal cells, especially actively proliferating cells, thus induce various complications such as bone marrow suppression, damage on gastrointestinal mucosa, and hair loss. Thus, the biggest problem of anticancer chemotherapeutics is the absence of selectivity. Anticancer effect could be obtained since cancer cells respond more sensitively and are destroyed to anticancer chemotherapeutics, while normal cells are rapidly regenerated after destruction.

Another complication of anticancer chemotherapeutics is threat of infection that is due to their immunosuppressive effects. Most of the current anticancer chemotherapeutics are classified into cytotoxic anticancer agents, while the rest of them include hormonal anticancer agents and biological response modifiers ARM) such as interferons and interleukin-2. Part of the biological response modifiers may be classified as immunotherapeutic agents.

Brief explanation on immunotherapeutic agents is as follows.

Human has an immune system that protects itself from harmful materials present both inside and outside the body. Immune system is composed of 2 mechanisms. One is cellular immunity where immune cells, such as macrophages and lymphocytes, are involved. The other is humoral immunity where antibodies are involved.

Abrogation in the cellular immunity is related to cancer development.

Immunotherapy is a method that-kills or inhibits the growth of cancer cells by inducing recovery or potentiation of the immune function that recognizes and discriminates cancer cells as antigens. Immunotherapy is divided into active, passive, and indirect ones.

Active immunotherapy is then subdivided into specific and non-specific ones. The latter is a method that non-specifically increases host immune functions using immunopotentiators such as Mycobacterium bovis BCG, while the former is a method that potentiates, immune response to-cancer-cells via vaccines against tumor antigens.

Meanwhile, passive immunotherapy contains humoral immunotherapy, such as monoclonal antibody, and cellular immunotherapy such as tumor infiltrating lymphocyte or lymphokine-activated killer cell (LAK). Monoclonal antibodies may be used as bound forms to anticancer agents or radioisotopes.

Indirect immunotherapy includes methods that inhibit cell growth factors or angiogenesis factors. In advanced cancers, the effect of immunotherapy has not been demonstrated either in immunotherapy alone or in combination with anticancer chemotherapy. Thus, immunotherapy is being used for treatment of early cancers by local administration.

Recently, the development of anticancer agents, which induce apoptosis, is actively being performed. Apoptosis is a cell death pathway occurring, in both physiological conditions, such as development and differentiation processes and pathological conditions such as cell damage and microbial infections.

The biochemical changes during apoptosis have been actively studied during the last decade. One of the breakthroughs was from the study in Caenorhabditis elegans. Ced-3, ced-4, and ced-9 genes are involved in the apoptosis pathway that occurs during the development of C. elegans. Among them, ced-3 and ced-4 are genes are involved in cell death, while ced-9 is a cell survival gene that protects an inappropriate apoptosis. The mammalian homologs of these ced genes were also found. Ced-3 homologs are caspases and are activated during apoptosis. Ced-4 homolog is apoptotic protease-activating factor 1 (Apaf1). Apaf1 is activated by cytochrome C release from mitochondria and induces the activation of other caspases. Ced-9 homolog is bcl-2 which was known to inhibit apoptosis.

As described above, various consecutive caspases has been found as ced-3 homologs. Caspases cleave specific aspartate residues in substrate proteins.

Apoptosis-inducing stimuli from outside cells are divided into 2 categories according to death receptor dependency. The death receptors for apoptosis include Fas, tumor necrotizing factor receptor 1, (TNFR1), TNF-related apoptosis-inducing ligand (TRAIL), TNF-receptor-related apoptosis-mediated protein (TRAMP), and nerve growth factor (NGF).

Death receptor-independent apoptosis stimuli include ultraviolet ray, gamma irradiation, heat shock, ceramides, anticancer agents, reactive oxygen species, viral infections, and removal of growth factors.

In the presence of these stimuli, small subunits in the c-termini of the initiator caspases are primarily removed by autocatalytic activity and the caspases are activated into active ones. Sequential proteolytic cascade is started by activated initiator caspases, which then induce proteolytic cleavage of other caspases. Consequently, classical morphological and biochemical changes of apoptosis occur when effector caspases are activated and act on cell death substrates.

Apoptotic sells die with characteristic morphological changes such as nuclear chromatin condensations, plasma membrane blebbing, apoptotic body formation, cytoskeleton change, and DNA fragmentation.

In death receptor-dependent pathway, the stimulus to the death receptor is transduced to pro-caspase 8 via an adaptor molecule, Fas-associated death domain (FADD). FADD activates caspase 8, which again activates effector caspases (such as caspase 6 and caspase 3) that acts on death substrates, resulting in cell death.

Meanwhile, death receptor-independent stimuli act directly on mitochondrial cytochrome C release from the inner membrane. The released cytochrome C activates Apaf1. And, consequently, apoptosome (a protein complex, Apaf1-cytochrome C-pro-caspase 9) is formed. Then pro-caspase 9 is activated that again activates effector caspases (caspase 3, caspase 7, and caspase 6) resulting in apoptosis.

Meanwhile, Bcl-2 is a well-known anti-apoptotic protein. There are about 15 proteins that have similar amino acid sequences to Bcl-2, which are called Bcl-2 family. Proteins belonging to Bcl-2 family have at; least 1 of Bcl-2 homology domains (H1 to BH4). However, not all bcl-2 family proteins inhibit apoptosis.

Bcl-2 family proteins are classified into anti-apoptotic and pro-apoptotic ones. Interactions between these 2 group proteins result in either induction or inhibition of apoptosis. For example, a typical anti-apoptotic (thus helping cell survival) protein Bcl-XL inhibits apoptosis by preventing structural change of Apaf1 protein. This structural change helps Apaf1 binding to pro-caspase 9. On thee other side, Bik, a pro-apoptotic protein, suppresses this anti-apoptotic function of Bcl-XL.

Anti-apoptotic proteins such as Bcl-2 and Bcl-XL are known to inhibit apoptosis by suppressing the cytochrome C release from mitochondria. These 2-proteins contain, at least, BH1 and BH2 domains.

Meanwhile, pro-apoptotic proteins of Bcl-2-family contain Bax subfamily that includes Bax, Bak, and Bok (all of which are structurally similar to Bcl-2), and BH3 subfamily. BH3 subfamily proteins, such as Bik, act as antagonists to anti-apoptotic proteins such as Bcl-XL and induce apoptosis.

Anti-apoptotic proteins and pro-apoptotic proteins may form heterodimers, which maintains a balance in apoptosis.

Thus, Bcl-2 family proteins are very important in controlling death receptor-independent apoptosis. Therefore, the main target of the death receptor-independent apoptotic signals may include Bcl-2 family proteins.

The goal of most anticancer agents-is the induction of apoptosis of cancer cells. Present anticancer agents can also induce apoptosis, however, without a specific target. Anticancer agents, with apoptosis-regulating factors as specific targets, are now being developed.

The examples include Aptosyn (Cell Pathway Inc., Horsham, Pa., USA) that selectively stimulates the apoptosis of abnormal cells by inhibiting cyclic GMP phosphodiesterase and G-3139 (Genta Inc., Lexington, Mass., USA) that decreases the amount of Bcl-2 protein in cancer cells via inhibition of its mRNA synthesis.

Meanwhile, Mycobacteriinm ulcerans is a slow-growing mycobacterium that induces necrotizing, skin disease named Buruli ulcer. The slow-growing mycobacteria family also contains Mycobacterium tuberculosis, Mycobacterium leprae, and Mycobacterium marinum. These slow-growing mycobacteria, except Mycobacterium ulcerans, maintain, their virulence through their capability of surviving and growing inside human macrophage and thus present for a long time in human body. They also induce strong immune and inflammatory responses that are due to the presence of indigestible lipids in cell walls. Mycobacterium ulcerans, which has similar genetic background to these mycobacteria on ribosomal RNA sequence level, does not have these properties. Mycobacteriumn ulcerans has been thought to produce a spreading molecule, a kind of toxin, which has low immunogenicity. The toxin has been presumed not to be a protein toxin since it does not induce strong immune responses.

K. George and P. Small et al. (Rocky Mountain Laboratories, National Institute of Health) isolated, purified, and characterized a toxin of Mycobacterium ulcerans. They found that the toxin is not a protein but a kind of lipid [Infect. Immun., 66, (1998) 587-593]. Through purification and structural analyses, they demonstrated that it is a small lipid molecule containing polyketides and named it a mycolactone [Science, 283, (1999) 854-857].

K. George et al. revealed that mycolactone induces G1 cell cycle arrest and cytopathic effects such as detaching of cells from culture plates and cell rounding-up in murine L929 cell line. They also reported that mycolactone induces G1 cell cycle arrest within 48 hours and apoptosis: with prolonged treatment in murine L929 and J779 cell lines [K. George et al., Infect. Immun., 68, (2000) 877-883].

However, it is not known whether mycolactone acts as an effective anticancer agent through these mechanisms against cancer cells.

Meanwhile, Rb protein, which regulates excessive cell proliferation by inhibiting G1 to S progression in the cell cycle, is a typical molecule against apoptosis [Bartkova J. et al., Cancer Res., 56, (1996) 5475-5483].

Rb protein prevents excessive cell proliferation, and this function of Rb protein depends on its phosphorylation status. That is, hypophosphorylated Rb protein suppresses cell proliferation by inhibiting S phase entry and thus inducing G1 arrest through binding with E2F, an S phase transcriptional activator.

On the other hand, when Rb protein is hyperphosphorylated, which is unable to bind E2F proteins, cells proliferate through induction of various S phase gene expressions by free E2F proteins [Li Y J. et al., Oncogene, 11 (1995) 59700(; Weinberg R A, Cytokines Mol Ther., 2, (1996) 105-110].

The hyperphosphorylation of Rb protein occurs in the presence of cyclin dependent kinases (hereinafter, CDK). Again, CDK inhibitors are maintaining the balance of cell growth by regulating CDK activity [Kawamata N. et al., Cancer, 77-(1996) 570-575]. Thus, Rb protein regulates cell growth at the G1 phase when cells are exposed to growth factors.

On the other hand, Rb protein inhibits cell death in the presence of apoptosis-inducing factors. For example, several reports showed that the apoptotic cell death, induced by p53 protein overexpression or irradiation, is suppressed by Rb protein [Haas-Kogan D A. et al., EMBO, 14, (1995) 461-472; Haupt Y. et al., Oncogene, 10 (1995) 1563-1571]. Therefore, apoptosis-inducing anticancer agents might need a molecule(s) that decreases the expression of Rb protein.

### DETAILED DESCRIPTION OF THE INVENTION

The inventor found that the mycolactone-inducing cancer cell death was more effective in cancer cells without Rb expression.

After all, the first object of this invention is to provide mycolactone as an anticancer agent, which selectively destructs cancers in which Rb proteins are not expressed.

The second object of this invention is to provide inhibitors of Rb proteins expression, including an antisense Rb oligonucleotide, which sensitize cancer cells to mycolactone.

The third object of this invention is to provide an anticancer agent against Rb-positive cancers comprising both mycolactone and the inhibitors of Rb protein expression, including an antisense Rb oligonucleotide, through the mechanism described above.

This invention provides an apoptosis-inducing anticancer agent(s), against various types of cancers, comprising mycolactone, a toxin of Mycobacterium ulcerans that causes Buruli ulcer, which is reported to induce apoptosis in normal cell lines.

This invention provides an anticancer agent(s) that induces selective apoptosis in cancers in which Rb protein is not expressed.

This invention also provides inhibitors, which suppress Rb protein expression These Rb inhibitors, including an antisense Rb oligonucleotide comprising nucleotide sequence No. 3, increase the apoptosis-inducing activity of mycolactone even in Rb-positive cancer cells. Thus, this invention also provides an anticancer agent(s) selectively sensitive to Rb-positive cancer cells, comprising both mycolactone and the inhibitors of Rb proteins expression including an antisense Rb oligonucleotide.

Mycolactone showed a cell death effect on various types of cancers such as those of breast, bladder, skin, stomach, liver, colon, and oral cavity, lymphoma, and leukemia through induction of apoptosis. The effect of mycolactone was more effective in Rb-negative cancer cell line. And, even in Rb-positive cancer cell line, which is resistant to apoptosis, mycolactone-induced apoptosis could be obtained by transfecting antisense Rb oligonucleotide through the inhibition of Rb protein synthesis.

Therefore, the anticancer effect could be obtained with mycolactone only or in combination with antisense Rb oligonucleotide in Rb-negative cancer cells or in Rb-positive cancer cells, respectively.

The component(s) of this invention for clinical treatment of cancers can be used after preparation, according to conventional pharmaceutical methods, such as addition of polymers that is one of the pharmaceutically allowed carriers Preparations for oral administration is acceptable such as pills, tablets, capsules, liquid formulations, and suspensions. However, it is the most desirable to administrate the drug by local or systemic injections.

Dosage of the preparation of this invention for anticancer therapy depends on sex, age, type and seventy of cancers, and presence of coimplication(s). Generally, the daily dosage is 3 to 6 mg/kg and desirably 4 to 5 mg/kg.

### BRIEF DESCRIPTION OF THE DRAWINGS

FIGS. 1a, 1b, 1c, 1d, 1e, and 1f are photomicrographs showing cancer cell death by mycolactone treatment in skin cancer, stomach cancer, breast cancer, leukemia, bladder cancer, and hepatoma

FIG. 2 contains morphologic evidences of mycolactone-induced apoptosis in cancer cells by transmission electron microscopy.

FIG. 3 contains Western blot pictures showing the cancer cell death by mycolactone treatment is an apoptosis phenomenon.

FIG. 4 shows mRNA expression profile of apoptosis-related genes in cancer cells by mycolactone treatment.

FIG. 5 contains antisense Rb oligonucleotide (shown below as Antisense Rb) designed to prevent the transcription of human Rb gene, sense Rb nucleotide (shown above as Sense. Rb) used for control experiment, and the target regions of these oligonucleotides on human mRNA sequence (shown in the middle as Rb mRNA).

FIG. 6 is a Western blot picture showing the decrease of Rb protein expression in SNU475 (an Rb-positive cancer cell line) transfected with antisense Rb oligonucleotide.

FIG. 7 shows apoptosis phenomena occurred in SNU475 (an Rb-positive cancer cell line) after the treatment with antisense (right panels shown as Antisense Rb) or sense Rb oligonucleotide (left panels shown as Sense Rb), of which sequences are described in FIG. 5, followed by mycolactone treatment.

FIG. 8 shows the in vivo anticancer effect of mycolactone in nude mice model.

FIG. 9 shows the anti-angiogenesis effect of mycolactone by tube formation experiment.

### DETAILED DESCRIPTION OF THE PREFERRED EMBODIMENT

The following will be more detailed explanation of the present invention by examples.

These examples are only to explain the present invention and embodiments of the present invention are not limited only to the above, and it is evident that it can be diversely modified by a person who has ordinary knowledge in the appropriate field, within the technical idea of the present.

### EXAMPLE 1

### Cancer Cell Lines Cell Culture; and Observation of Mycolactone-Induced Cancer Cell Death Under Light Microscope

Cancer cell lines used for the experiment was as follows; 2 skin cancer cell lines (Malme3M and SK-Mel-24), 1 breast cancer cell line (MDAMB231), 1 leukemia cell line (MOLT4), 1 stomach cancer cell line (SNU1); 1 bladder cancer cell line (TCCSUP), 8 hepatoma cell lines (SK-Hep1, Hep3B, SNU182, SNU387, SNU398, SNU449, SNU475, and HepG2), 2 colon cancer cell lines (HT-29 and DLD-1), and 1 oral cavity cancer cell line (SCC-15). Cancer cells were cultured in RPMI1640 media containing 10% fetal bovine serum, penicillin (10 unit/ml), and streptomycin (100 μg/ml) in 75 cm² flask at 37° C. in the presence of 5% CO₂.

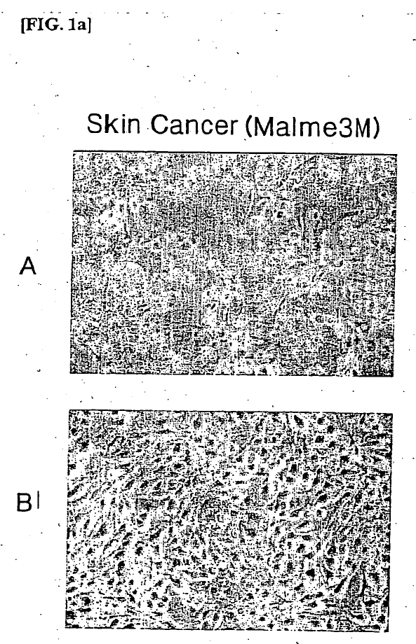

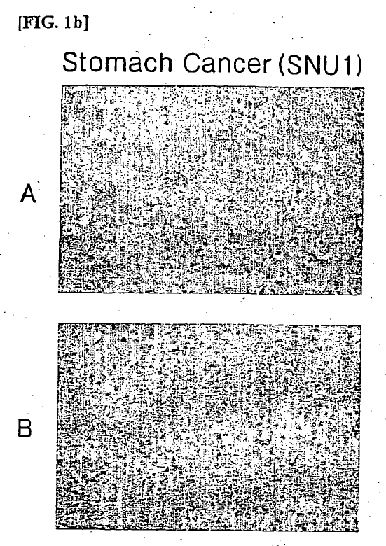

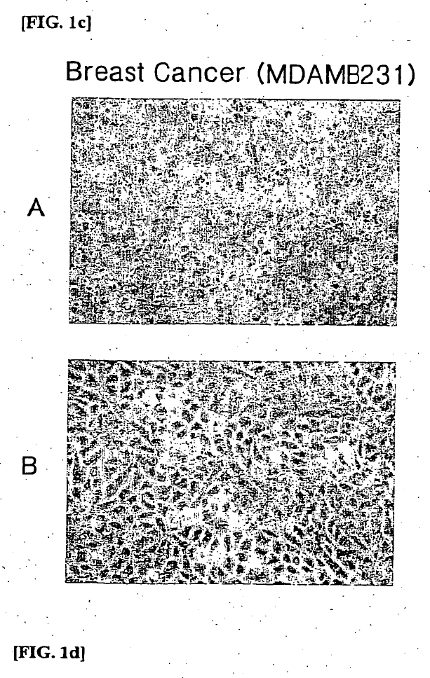

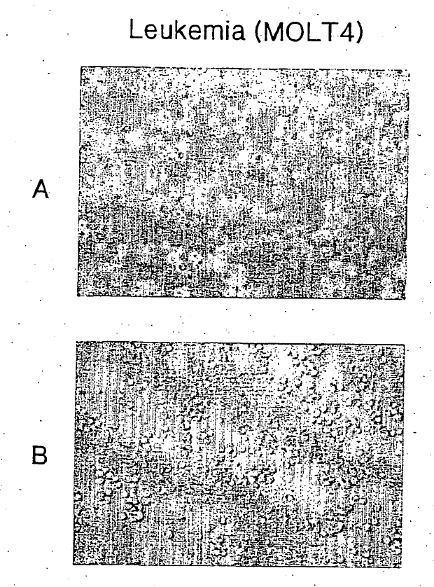

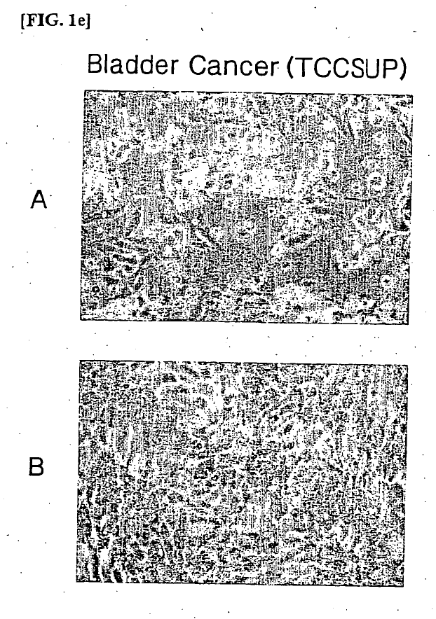

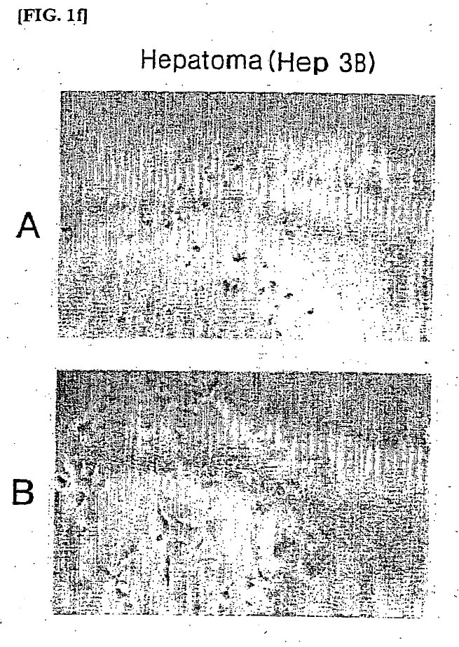

After cultivation of cancer cells (5×106) in 6-well plate for 24 hours, mycolactone (final 1 μg/ml) was added and the cell morphology was observed after 72 hours under light microscope. Cancer cells became round up and died after mycolactone treatment. Significant cell death was observed in cancer cells treated with mycolactone (A) compared to those not-treated (B) (FIGS. 1a, 1b, 1c, 1d, 1e, and 1f).

FIGS. (1a to 1f) show the results in skin cancer (Malme3M), stomach cancer (SNU1), breast cancer (MDAMB231), leukemia (MOLT4), bladder cancer (TCCSUP), and hepatoma (Hep3B). The morphological change of colon and oral cavity cancers is not shown.

### EXAMPLE 2

### Cancer Cell Death Effect of Mycolactone via Apoptosis Induction

Morphological change of Hep3B cancer cells was observed by transmission electron microscopy (hereinafter, TEM) Cancer cells (5×10⁶) were cultured and treated with mycolactone (1 μg/ml). Cell were collected after 24 hours and fixed with 2.5% glutaraldehyde. The sample was treated with OsO₄, dehydrated with ethanol, and embedded to Epson resins. After staining with uranyl acetate and lead citrate, each section was observed with TEM (Hitachi 7100B, Japan).

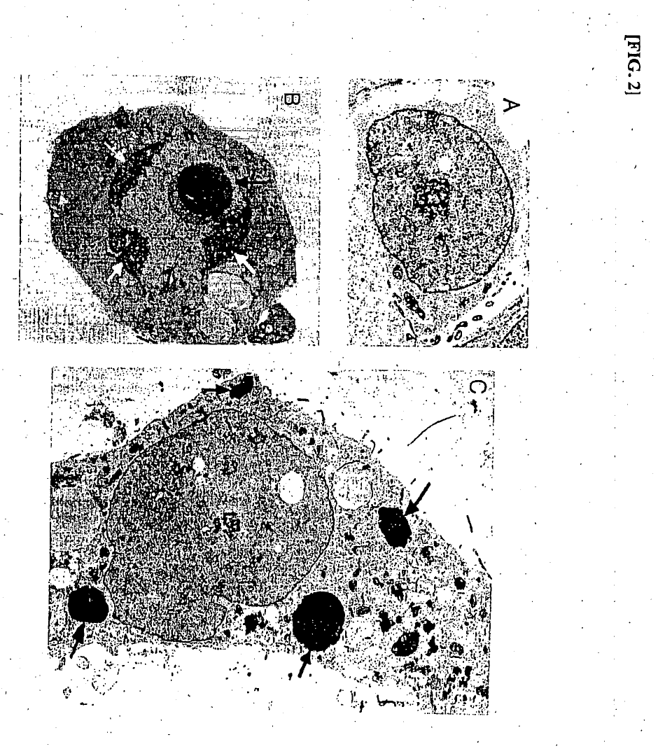

The result shows typical apoptosis morphologies in mycolactone-treated case (B) such as chromatin condensations (white arrows), formation of apoptotic bodies (black arrows), and ingestion of apoptotic bodies (black arrows) by neighboring cells (C) (FIG. 2). The ingested apoptotic-bodies are the ones excluded from dead cells. These findings were not found in not treated case (A).

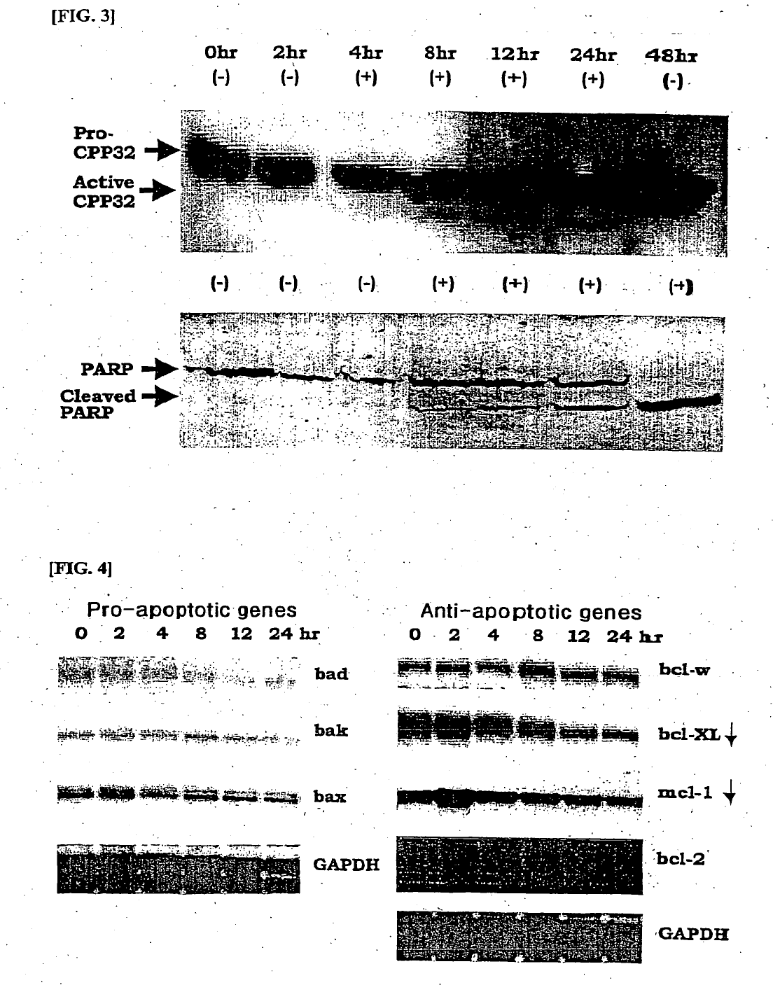

CPP32 caspase activation and following cleavage of poly-ADP-ribose polymerase, typical biochemical phenomena of apoptosis, were examined by Western-blotting. Hep3B cancer cells (5×10⁶) were cultured and treated with mycolactone (final 1 μg/ml). Cells were collected after 2, 4, 8, 12, 24, or 48 hours and Western blot was performed with anti-CPP32 antibody (Oncogene, Mass., USA) or anti-poly-ADP-ribose polymerase antibody (Enzyme Systems Products, CA, USA). Protein preparation was performed as follows. Cells were suspended in 400 μl of lysis buffer (125 mM Tris-HCl [pH 6.8], 20% glycerol, 2% SDS, and 10% β-mercaptoethanol), vortexed for 30 seconds, and kept at 95° C. for 5 minutes Cell lysates were separated on a 10% SDS-polyacrylamide gel and transferred to nitrocellulose membrane. Antibody reaction was performed with the antibodies describe above. CPP32 caspase activation was started at 4 hours and maintained until 24 hours, while the cleavage of poly-ADP-ribose polymerase was started at 8 hours after mycolactone treatment. The poly-ADP-ribose polymerase was completely cleaved after 48 hours (FIG. 3). Pro-CPP32 and Active CPP32 as shown on left are CPP32 caspase (the caspase 3) before and after activation, respectively; PARP is poly-ADP-ribose polymerase; cleaved PARP is cleaved poly-ADP-ribose polymerase.

These results provided morphological and biochemical, evidences that mycolactone-induced cancer cell death is an apoptosis phenomenon.

### EXAMPLE 3

### Apoptosis-Regulating Genes of which Expressions are Affected by Mycolactone

To find target genes of mycolactone, mRNA transcription levels of bcl-2 family genes were examined. Total RNA was prepared from mycolactone-treated Hep3B cells and the expression of 7 genes belonging to bcl-2 family by ribonuclease protection assay (RPA). Total RNA was prepared with RNeasy minikit (Qiagen Inc., Chatsworth, Calif.) as described below. Mycolactone treated cancer cells were collected and washed with PBS. Cells were suspended in lysis buffer containing β-mercaptoethanol. Cells were passed through a 20-G syringe for more than 5 times. Equal volume of 70% ethanol was added and the suspension was mixed well. The suspension was applied to RNeasy mini spin column. The column was centrifuged at 10,000 rpm for 15 seconds and washed 2 times with washing buffer plus PRE buffer. RNA attached to the column was eluted with RNase-free distilled water. RNA was stored at −70° C. before use.

The mRNA expression profile of bcl-2 family genes was examined by RPA using multi-probe RNase Protection Assay System (PharMingen, CA, USA) with the following procedures. Specific RNA probe labeled with radioisotope is synthesized and used for hybridization with the RNA prepared from each sample. After removal of single-stranded RNA that is not hybridized with the probe and the residual riboprobe, the sample is electrophoresed on a denaturing polyacrylamide gel. After autoradiography, the mRNA expression was analyzed through measuring the density of hybridized bands.

RPA is a 3-Step Procedure

1). Synthesis of probe: Probe is synthesized by incubating transcription mixture solution (10 μ[α-³²P]UTP, 1 μl GACU pool, 2 μl DTT, 4 μl 5× transcription buffer, 1 μl RPA template set, 1 μlA T7 polymerase) at 37° C. for 1 hour. The reaction was stopped by adding 2 μl of DNase. Probe synthesis was completed by phenol treatment and ethanol precipitation The precipitated probe was dissolved in 50 μl of hybridization buffer.

2) RNA preparation and hybridization: Total RNA prepared (10-20 μg) was kept at −70° C. for 15 minutes and dried completely in vacuum evaporator. Hybridization was performed by the following reactions; addition of 8 μl of hybridization buffer, vortexing and brief centrifuge; addition and mixing of 2 μl of probe diluted at about 3×10⁵ cpm/μl; addition of mineral oil. Hybridization mixture was kept briefly at 90° C., then incubated at 56° C. for 12 to 16 hours. Hybridization was completed by incubating the mixture at 37° C. for 15 minutes.

3) RNase treatment, electrophoresis, and autoradiography: RNase mixture (100 μl) was added to the hybridization mixture and the non-hybridized RNA was removed by incubating at 30° C. for 45 minutes. RNase digestion was terminated by adding proteinase K mixture solution. After phenol treatment and ethanol precipitation, the sample was dried. The sample was nixed with 5 μl of 1× loading buffer, heated at 90° C. for 3-minutes, kept on ice, electrophoresed ort a denaturing polyacrylamide gel, dried, and exposed to X-ray film.

The mRNA expression profile of mycolactone-treated Hep3B cells showed no change in pro-apoptotic genes (bad, bak, and bax) until 24 hours after treatment (FIG. 4, left panel). The decrease of mRNA expression of bcl-XL, one of the anti-apoptotic genes, was observed starting at 8 hours until 24 hours. The mRNA expression of mcl-1, another anti-apoptotic gene, was increased transiently at 2 hour and decreased slowly after 4 hours, and the decrease was maintained until 24 hours after treatment. No change was found in case of bcl-w by mycolactone. The bcl-2 showed very low mRNA expression without significant change (FIG. 4, right panel).

These results suggested that the mechanism of mycolactone-induced apoptosis involves the down-regulation of anti-apoptotic bcl-XL and mcl-1 genes.

### EXAMPLE 4

### Synthesis of Antisense Rb Oligonucleotide

Antisense Rb oligonucleotide (sequence No. 3) that inhibits Rb gene expression was synthesized based on the human cDNA sequence of Rb gene (sequence No. 2). In this invention, antisense Rb oligonucleotide was synthesized with the protein initiation codon-region of Rb mRNA as a target. To inhibit the destruction by the intracellular nucleases, oligonucleotides with phosphorothioate backbone were synthesized.

For the control experiment, sense Rb oligonucleotide (sequence No. 1) was synthesized by the same method as described above.

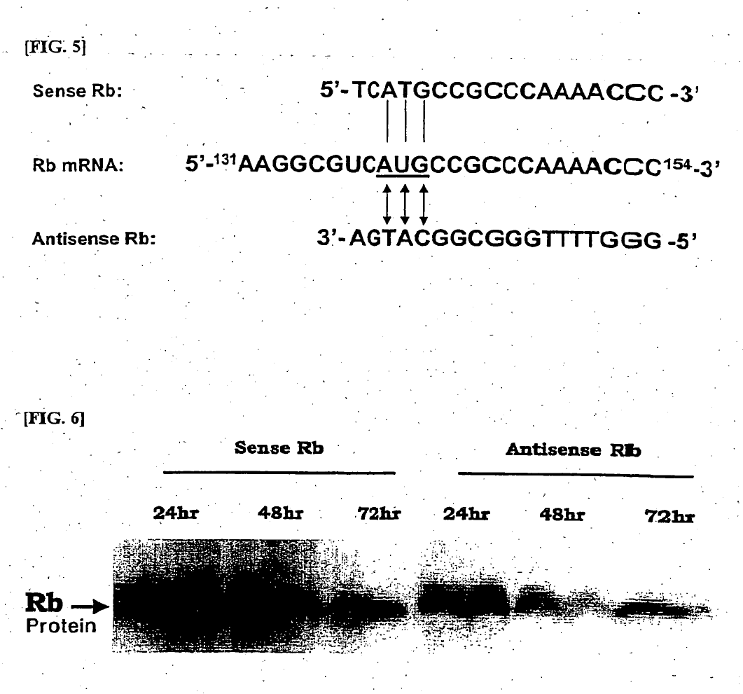

The sequence of each oligonucleotide is shown in FIG. 5.

### EXAMPLE 5

### Inhibition of Rb Protein Synthesis by Transfection of Antisense Rb Oligonucleotide to Rb-Positive Cancer Cells

An Rb-positive cancer cell SNU475 was cultured overnight in 6-well plate (5×10⁶ cancer cells per well) with RPMI1640 medium. Sense or antisense Rb oligonucleotide, as described in Example 4, was transfected to the cultured cancer cells using Lipofectamine-PLUS (Gibco BRL, Grand Island, N.Y.) with the following procedures. Sense or antisense Rb oligonucleotide was diluted (final 1 uM) in fetal bovine serum-free RPMI1640 medium. PLUS reagent (Gibco BRL, NY, USA) was added, mixed well, and incubated at room temperature for 15 minutes. During this incubation, Lipofectamine was diluted in fetal bovine serum-fee RPMI1640 medium in separate test tubes. After 15 minutes, 2 solutions were mixed well and incubated at room temperature for 30 minutes for induction of oligonucleotide-Lipofectamine complex formation. During this incubation, the medium in overnight culture of the cancer cells was changed with fresh fetal bovine serum-free RPMI1640 medium. The solution containing oligonucleotide-Lipofectamine complex was carefully dropped on each culture plate and incubated at 37° C. for 3 hours. RPMI1640 medium containing fetal bovine serum was added and cells were cultured overnight.

Nuclear protein fraction was prepared to examine the Rb expression in SNU475 cell line transfected with sense or antisense Rb oligonucleotide by Western blotting. After decanting the medium and adding cold PBS, cells were collected from the culture plate, by scraper. Collected cells were centrifuged, resuspended in 400 μl of cold buffer A (10 mM Hepes-KOH [pH 7.9], 1.5 mM MgCl₂, 10 mM KCl, 0.5 mM DTT, 0.2 mM PMSF, 0.1% NP-40), and kept on ice for 30 minutes. The mixture was vortexed for 10 seconds and centrifuged. Cold buffer C (20 mM Hepes-KOH [pH 7.9], 25% glycerol, 420 mM NaCl, 1.5 mM MgCl2, 0.2 mM EDTA, 0.5 mM DT, 0.2 mM PMSF) was added, well suspended, and kept on ice for 30 minutes. Cell debris was removed by spin down the mixture at 4° C. for 2 minutes. The protein concentration in the supernatant was determined and used for Western blotting.

Nuclear protein preparation (40 μg) of each cancer cell line was electrophoresed on a 4-20% gradient SDS-polyacrylamide tris-glycine gel (Novex, Calif., USA). After electrophoresis the gel was removed from the apparatus and applied to Western blot apparatus (Novex, Calif., USA). Protein was transferred to nitrocellulose membrane at 30V for 2 hours in the presence of transfer buffer (12 mM Tris, 96 mM glycine, 20% methanol, pH 8.3). The nitrocellulose membrane was incubated in a blocking solution (PBS containing 5% non-fat milk and 0.02% sodium azide) for 30 minutes. Mouse anti-human Rb monoclonal antibody (2 μg/ml, PharMingen, CA, USA) was added and the solution was incubated for 1 hour. Nitrocellulose membrane was washed once with PBS, twice with PBST, and finally once with PBS. Nitrocellulose membrane was soaked in blocking solution (PBS containing 5% non-fat milk). Anti-mouse immunoglobulin G antibody conjugated with horseradish peroxidase (HRP) was added and the solution was incubated for 30 minutes.

After washing the membrane with PBS and PBST, light reaction was performed using enhanced chemiluminescence (ECL) reagent. The membrane was exposed to X-ray film for appropriate time.

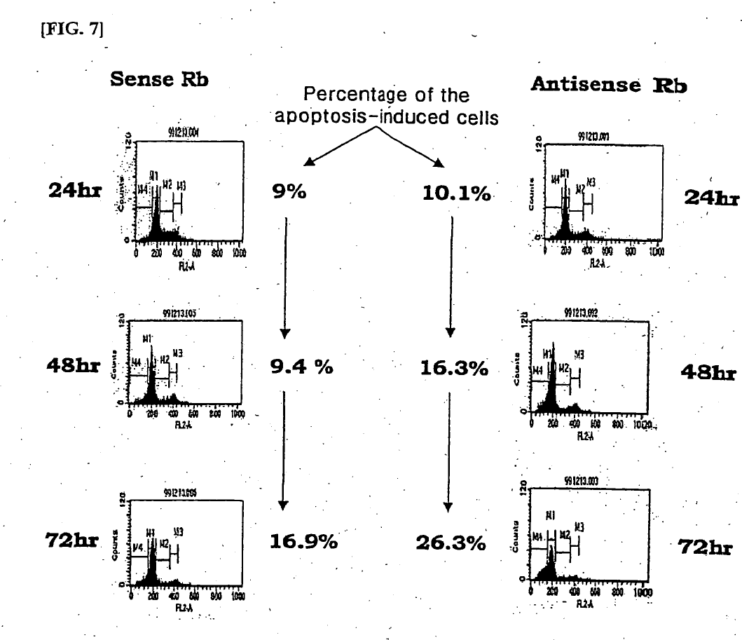

Significant time-dependent decrease of Rb protein expression was found in transfectants with antisense Rb oligonucleotide compared to that in transfectants with sense Rb oligonucleotide (FIG. 6).

These results confirmed that the antisense Rb oligonucleotide of this invention effectively inhibits the Rb protein expression.

### EXAMPLE 6

### Potentiation of Cell Death Effect in Rb-Positive SNU475 Cancer Cells by Mycolactone Treatment in Combination with Antisense Rb Oligonucleotide

Mycolactone was added to SNU475 cancer cells (5×10⁶) after transfection with sense or antisense Rb oligonucleotide. FACS analysis was performed after 24, 48, or 72 hours to examine the anticancer effect with following procedures.

Cells were washed with 450 μl of PBS and suspended well. Cells were fixed with 1 ml of 70% ethanol for 30 minutes, centrifuged, resuspended in 1 ml of FACS buffer (PBS containing 10 μg/ml RNase and 50 μg/ml propidium iodide), and kept at 37° C. for 30 minutes. FACS analysis was performed immediately with FACStar Instrument (Beckton-Dickinson Immunocytometry Systems, Los Angels, Calif.).

There was significant difference between transfectants with sense and antisense Rb oligonucleotides. That is, mycolactone induced cell death in 9.0%, 9.4%, and 16.9% and in 10.1%, 16.3% and 26.3%, at each time, of total transfectants with sense Rb oligonucleotide or antisense Rb oligonucleotide, respectively (FIG. 7). The partial (26.3%) cell death in transfectants with antisense Rb oligonucleotide even after 72 hours might be due to residual Rb protein present even after antisense transfection (as-shown in FIG. 6), which seemed to slightly inhibit mycolactone-induced cancer cell death.

These results showed that the antisense Rb oligonucleotide transfection sensitizes cancer cells to mycolactone, resulting in the increase of apoptotic population even in Rb-positive cancer cells.

### EXAMPLE 7

### An In Vivo Anticancer Effect of Mycolactone in Nude Mice

Hep3B human hepatoma cells were transplanted to nude mice and tumor growth was induced for 2 to 3 weeks. Then, mycolactone was injected to the tumor tissue by 4-3 days method, that is, injection for 4 days and rest for 3 days. PBS solution (50 μl) with or without mycolactone (20 μg) was injected to tumor of treatment (T) or control (C) mouse, respectively. The tumor volume was estimated by measuring both long and short diameters. The tumor volume at the day of first injection was 101.3 mm³ (C) or 105.9 mm³ (T).

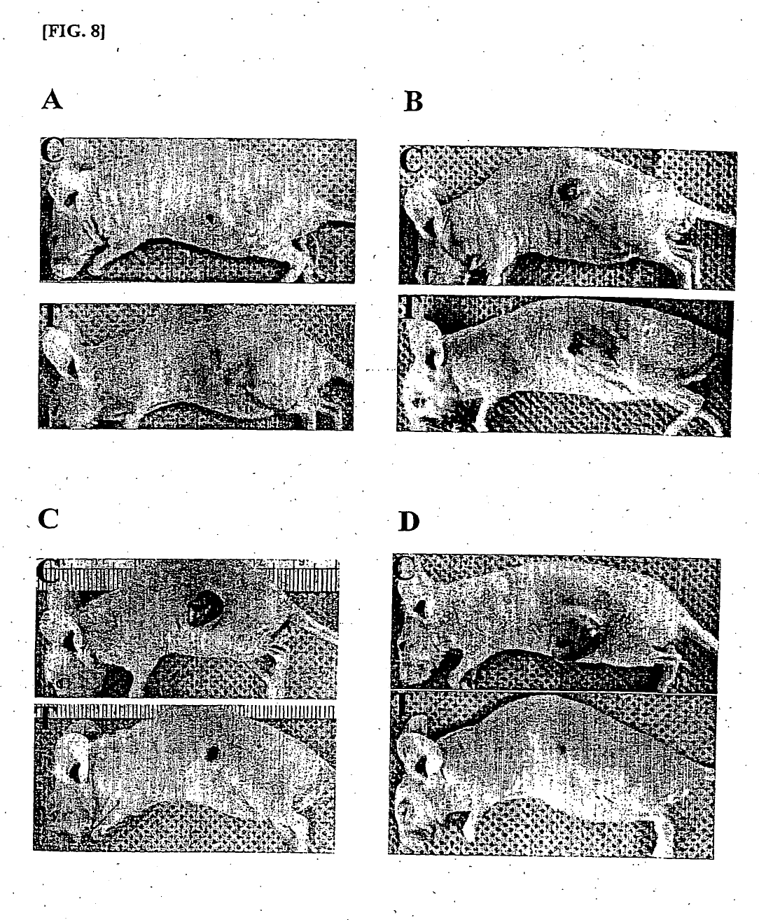

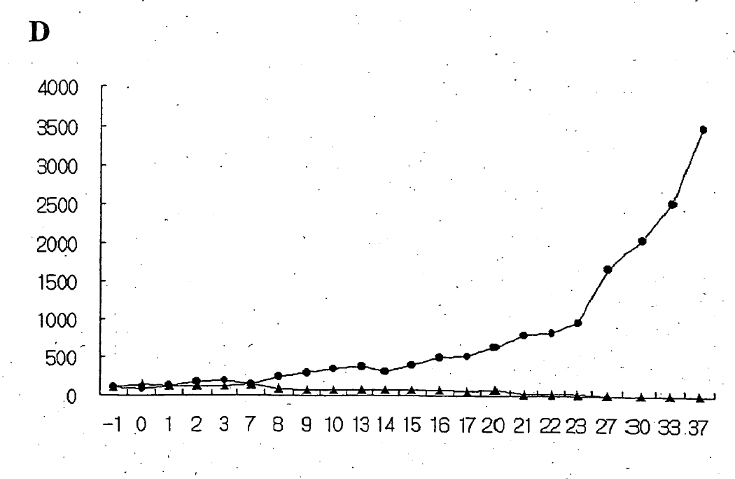

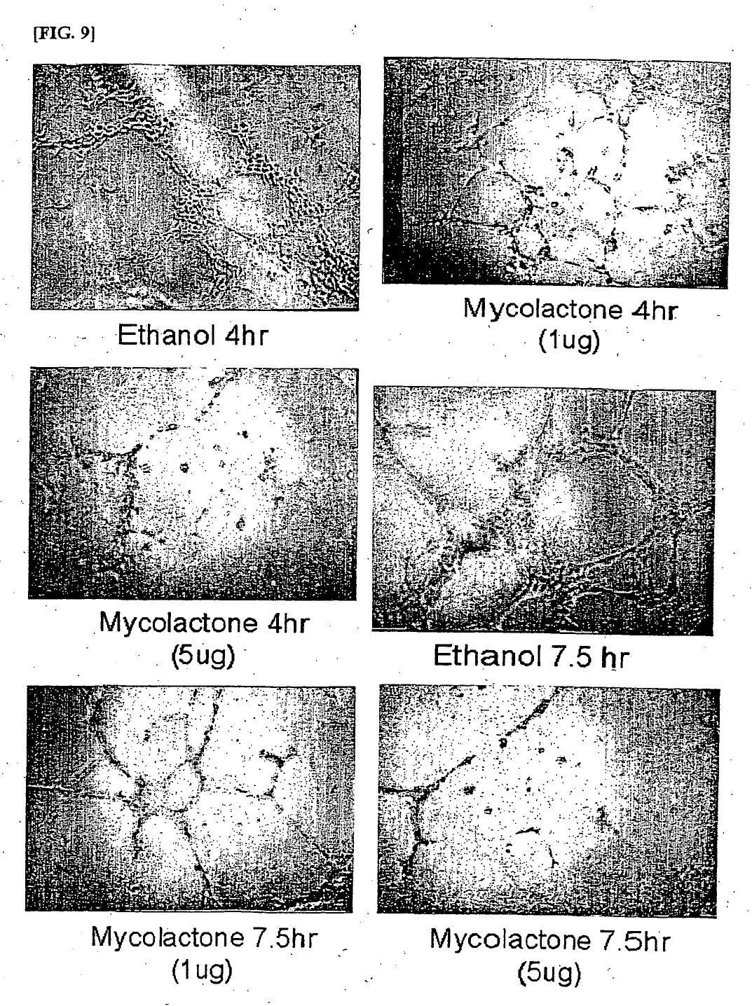

At first week, no significant difference was found between C and T mice. At second week, significant tumor growth (310.7 mm) was observed in C mouse while the tumor shrinkage (63.8 mm³) was found in T mouse with central necrosis. At third week, the tumor volume of C mouse was greatly increased (800.6 mm³), while that of T mouse was more reduced (20.8 mm³) with crust formation at the center. At fourth week, the tumor of C mouse became very huge (1676.2 mm), while that of T mouse was disappeared with a small wound, which was healed at fifth week (FIG. 8A). The time sequence change of tumor volume is shown in FIG. 8B. The X-axis is time in days, while the Y-axis is tumor volume in mm³. Tumor volumes in C or T mouse are depicted in filled circles or triangles, respectively. At day 37, the C mouse bearing a huge tumor mass of 3501.7 mm³ was sacrificed. The T mouse was healthy until the day 45 when it was sacrificed and was confirmed not to have any tumor tissue inside the body.

These data showed that mycolactone also has a very strong anticancer effect in vivo.

### EXAMPLE 8

### An Anti-Angiogenesis Effect of Mycolactone

The in vivo anticancer effect of mycolactone shown in Example 7 was too strong to be explained only by its apoptosis-inducing activity. Therefore, tube formation experiment was performed to examine whether mycolactone inhibits angiogenesis, which can further explain the in vivo anticancer effect of mycolactone.

HUVEC (Human umbilical vein endothelial cell, American Type Culture Collection, Manassas, Va.) cells were maintained in HAM's F-12K nutrient mixture (Sigma, St. Louis, Mo.) with 10% fetal bovine serum and endothelial cell growth supplement (Sigma, St. Louis, Mo.). Cells were plated onto a 1% gelatinized plastic surface and incubated in the presence of 5% CO₂ at 37° C. Tube formation assay was performed using an In Vitro Angiogenesis Assay Kit. (Chemicon, Temecula, Calif.) with following procedures. A 50 μl of the Diluent Buffer-ECMatrix solution mixture was transferred to 96-well tissue culture plate and kept at 37° C. for 1 hour for solidification of the matrix solution. HUVEC cells were seeded onto the surface of the polymerized ECMatrix in each well and incubated in the presence of ethanol (control) or mycolactone (containing 1% of ethanol).

Tube formation was very clear in control cases (ethanol only) after 4 hour. However, in case of mycolactone treatment (1 μg), tube formation was inhibited after 4 hour and the inhibition was maintained until 7.5 hour. In case of higher amount of mycolactone treatment (5 μg), tube formation was almost completely inhibited after 4 hours. This anti-angiogenesis effect might be due to the apoptosis-inducing activity of mycolactone to vascular endothelial cells.

These results suggested that mycolactone inhibits angiogenesis, which is essential in tumor growth, and also provides an explanation of its strong in vivo anticancer activity shown in Example 7.

### INDUSTRIAL APPLICABILITY

According to the present invention, it is clear that the cancer cell death by the anticancer agent(s) of this invention, comprising mycolactone, is due to the apoptosis-inducing activity of mycolactone in cancer cells. And the mycolactone-induced apoptosis is, in part, due to the inhibition of mRNA expressions of bcl-XL and mcl-1 genes.

The anticancer effect of mycolactone is more striking in Rb-negative cancer cells than in Rb-positive ones. Besides, the Rb protein expression in Rb-positive cancer cells can be suppressed by inhibitor(s) such as antisense Rb oligonucleotide. Therefore, apoptotic cancer cell death by mycolactone can be increased in this condition. As a result, the anticancer effect of mycolactone, against Rb-positive cancers, can be increased when mycolactone is combined with an inhibitor(s)-of Rb protein expression such as antisense Rb, oligonucleotide. Mycolactone shows very strong anticancer effect in vivo as well as in vitro. The mechanisms of in vivo anticancer effect of mycolactone may include its anti-angiogenesis activity.

The anticancer agents of this invention can be app to various types of cancers such as those of breast, bladder, skin, stomach, liver, colon, and oral cavity, lymphoma, and leukemia and so on.

What is claimed are:

1. An anticancer agent which is characterized in comprising mycolactone.

2. The anticancer agent of claim 1 wherein the anticancer agent is specific to cancers in which Rb proteins are not expressed.

3. An anticancer agent comprising both mycolactone and inhibitors of Rb protein expression.

4. The anticancer agent of claim 3 wherein the Rb inhibitors comprise an antisense Rb oligonucleotide.

5. The Rb inhibitors of claim 4 wherein the antisense Rb oligonucleotide comprises nucleotide sequence No. 3.

6. The anticancer agent of claim 3 or 5 wherein the anticancer agent is specific to cancers in which Rb proteins are expressed.

7. The anticancer agent of claim 2 and 6 wherein the cancers include those of breast, bladder, skin, stomach, liver, colon, and oral cavity, lymphoma, and leukemia.


SEQUENCE LISTING (3 sequences)

SEQ ID NO: 1 - DNA, 18 bp, Artificial Sequence
  Sense retinoblastoma oligonucleotide
tcatgccgcccaaaaccc

SEQ ID NO: 2 - DNA, 4740 bp, Homo sapiens retinoblastoma 1
  mRNA (131)..(154)
  ref <302>: TITLE: GenBank
  ref <308>: DATABASE ACCESSION NUMBER: NM_000321
  ref <309>: DATABASE ENTRY DATE: 2000-10-31
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcacgagttgacctagatgagatgtcgttcactttactgagctacagaaaaacatacgaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatcgaagattatttttggatcatgataaaactcttcagactgattctatagacagttttgaaacacagagaacaccacgaaaaagtaaccttgatgaagaggtgaatataattcctccacacactccagttaggactgttatgaacactatccaacaattaatgatgattttaaattctgcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtataactttcccaggttctgtttatggccacatttaatatcttcagctctttttgtggatataaaatgtgcagatgcaattgtttgggtgagtcctaagccacttgaaatgttagtcattgttatttatacaagattgaaaatcttgtgtaaatcctgccatttaaaaagttgtagcagattgtttcctcttccaaagtaaaattgctgtgctttatggatagtaagaatggccctagagtgggagtcctgataacccaggcctgtctgactactttgccttcttttgtagcatataggtgatgtttgctcttgtttttattaatttatatgtatatttttttaatttaacatgaacacccttagaaaatgtgtcctatctatcttccaaatgcaatttgattgactgcccattcaccaaaattatcctgaactcttctgcaaaaatggatattattagaaattagaaaaaaattactaattttacacattagattttattttactattggaatctgatatactgtgtgcttgttttataaaattttgcttttaattaaataaaagctggaagcaaagtataaccatatgatactatcatactactgaaacagatttcatacctcagaatgtaaaagaacttactgattattttcttcatccaacttatgtttttaaatgaggattattgatagtactcttggtttttataccattcagatcactgaatttataaagtacccatctagtacttgaaaaagtaaagtgttctgccagatcttaggtatagaggaccctaacacagtatatcccaagtgcactttctaatgtttctgggtcctgaagaattaagatacaaattaattttactccataaacagactgttaattataggagccttaatttttttttcatagagatttgtctaattgcatctcaaaattattctgccctccttaatttgggaaggtttgtgttttctctggaatggtacatgtcttccatgtatcttttgaactggcaattgtctatttatcttttatttttttaagtcagtatggtctaacactggcatgttcaaagccacattatttctagtccaaaattacaagtaatcaagggtcattatgggttaggcattaatgtttctatctgattttgtgcaaaagcttcaaattaaaacagctgcattagaaaaagaggcgcttctcccctcccctacacctaaaggtgtatttaaactatcttgtgtgattaacttatttagagatgctgtaacttaaaataggggatatttaaggtagcttcagctagcttttaggaaaatcactttgtctaactcagaattatttttaaaaagaaatctggtcttgttagaaaacaaaattttattttgtgctcatttaagtttcaaacttactattttgacagttattttgataacaatgacactagaaaacttgactccatttcatcattgtttctgcatgaatatcatacaaatcagttagtttttaggtcaagggcttactatttctgggtcttttgctactaagttcacattagaattagtgccagaattttaggaacttcagagatcgtgtattgagatttcttaaataatgcttcagatattattgctttattgcttttttgtattggttaaaactgtacatttaaaattgctatgttactattttctacaattaatagtttgtctattttaaaataaattagttgttaagagtc

SEQ ID NO: 3 - DNA, 18 bp, Artificial Sequence
  Antisense retinoblastoma oligonucleotide
gggttttgggcggcatga